(19) United States
(12) Patent Application Publication
(10) Pub. No.: US 2022/0218621 A1
(43) Pub. Date: Jul. 14, 2022

(54) EXTRACELLULAR VESICLES FOR DELIVERING THERAPEUTIC OR DIAGNOSTIC DRUGS

(71) Applicants: UNIVERSITA' DEGLI STUDI DI MILANO, Milano (IT); FONDAZIONE IRCCS ISTITUTO NAZIONALE DEI TUMORI, Milano (IT)

(72) Inventors: Paolo CIANA, Milano (IT); Mariangela GAROFALO, Milano (IT); Alessandro Maria VILLA, Milano (IT); Vincenzo MAZZAFERRO, Milano (IT); Adriana MAGGI, Milano (IT)

(21) Appl. No.: 17/615,302

(22) PCT Filed: May 29, 2020
(86) PCT No.: PCT/IB2020/055113

§ 371 Date: Nov. 30, 2021

(30) Foreign Application Priority Data
May 31, 2019 (IT) 102019000007785

(51) Int. Cl. A61K 9/50; A61K 49/00; C12N 5/078

(52) U.S. Cl. CPC A61K 9/5068; C12N 5/0634; A61K 49/0034; A61K 49/0097

(57) ABSTRACT

Extracellular vesicles isolated from blood plasma of oncological patients for selectively delivering therapeutic or diagnostic drugs are provided. Methods for isolating extracellular vesicles from an isolated sample of blood plasma and for preparing an isolated sample of blood plasma from oncological patients are also provided.

### TECHNICAL FIELD OF THE INVENTION

The present invention applies to the medical field and, in particular, to tumor diagnosis or treatment.

Surgical therapy in oncology remains the most effective treatment for the eradication of solid tumors. The success of the therapy depends almost exclusively on the surgeon's ability to resect the tumor margins. This ability is currently not easily standardized and is entrusted to the experience and tactile/visual sensitivity of the surgeons themselves. The use of highly selective diagnostic compounds would allow the surgeon to visualize the margins of the tumor within healthy tissue during the surgical procedure either directly or through imaging methods.

The effectiveness of imaging methods, such as PET, MRI, and ultrasound in tumor diagnosis, depends on their detection sensitivity. Selective accumulation of contrast molecules (e.g. ICG, gadolinium, 18FDG, microbubbles) would make it possible to increase the signal-background noise ratio.

Over the past twenty years, many nanoparticles of different nature (liposomes, extracellular vesicles, biocompatible nanoparticles) have been suggested as pathotropic delivery system in the oncological field; however, few methods have achieved clinical practice, e.g. such as lipoplatin or liposomal doxorubicin.

Recently, the publications by M. Garofalo et al. (Journal of Controlled Release 2018 Aug. 10; 283:223-234; Journal of Controlled Release, 2019 Jan. 28; 294:165-175; Viruses, 2018 Oct. 13;10(10)) report studies on the effect and selectivity of oncolytic and paclitaxel viruses enveloped in extracellular vesicles in the treatment of lung cancer cells.

Although limited, these few examples of applications are significant, because these formulations have made it possible to significantly reduce the toxicity of chemotherapeutic drugs in the treatment of different types of tumor.

However, there are limitations in the systems suggested so far, the main ones of which are: 1) limited pathotropicity, 2) poor biocompatibility and 3) limited ability to deliver large molecules, typically represented by biological drugs.

International patent application WO 2019/077534 describes the isolation of exosomes from plasma and their possible therapeutic use, however without providing any practical examples of how the method can be carried out.

International patent application WO 2019/191444 describes the use of engineered exosomes through the transfection of nucleic acids which express a therapeutic protein for the delivery of therapeutic drugs to specific targets, in particular by virtue of growth factor gradients.

### SUMMARY OF THE INVENTION

The inventors of the present patent application have surprisingly found that it is possible to use extracellular vesicles isolated from the plasma of oncological patients to deliver diagnostic or therapeutic drugs selectively to tumor cells.

### BRIEF DESCRIPTION OF THE FIGURES

FIG. 1 (left panel) shows the results of the characterization of extracellular vesicles obtained according to the present invention;

FIG. 1 (right panel) shows the results of the dimensional analysis using the NTA technique;

FIG. 2A shows the results of in vivo imaging tests (left) and ex vivo tests (right) on rats after IV injection with extracellular vesicles derived from the plasma of an oncological patient loaded with Indocyanine green;

FIG. 2B shows the results of in vivo imaging tests (left) and ex vivo tests (right) on rats after IV injection with extracellular vesicles derived from the plasma of a healthy individual loaded with Indocyanine green;

FIG. 3A shows the results in rats after 18 from the IV injection of 50 μL of gadoteric acid;

FIG. 3B shows the results in rats after 18 from the IV injection of 50 μL of gadoteric acid loaded in extracellular vesicles according to the present invention;

FIG. 4 shows the results of fluorescence assays acquired by IVIS Lumina following the incorporation of ICG fluorescent dye into the extracellular vesicles of the invention incubated for minutes (left) or 12 hours (right) with the fluorescent molecule;

FIG. 5 shows the results of fluorescence assays acquired by IVIS Lumina following the incorporation of antibodies in the extracellular vesicles of the invention incubated for 10 minutes (left) or 12 hours (right) with the fluorophore-conjugated antibody Alexafluor647;

FIG. 6 shows the results of the chemiluminescence acquisition of dot blots on extracellular vesicles according to the invention incubated for 10 minutes (left, −) or 12 hours (right, +) with digoxygenin-conjugated oligonucleotides;

FIG. 7 shows the results of the experiments shown in Example 8;

FIG. 8 shows the results of the experiments shown in Example 9.

### OBJECT OF THE INVENTION

In a first object, the present patent application describes extracellular vesicles isolated from the plasma of an oncological patient comprising drugs having diagnostic or therapeutic tumor activity.

In a second and third objects of the invention, a method for isolating and a process for purifying extracellular vesicles from an isolated sample of plasma of an oncological patient are described.

A fourth object describes a method for loading drugs with diagnostic or therapeutic activity into extracellular vesicles isolated from the plasma of oncological patients.

A fifth object describes the medical use of the extracellular vesicles isolated from the plasma of oncological patients and loaded with a drug having diagnostic and/or therapeutic activity for the diagnosis and/or treatment of tumors.

A further object describes a method for the diagnosis and/or therapy of tumors comprising the administration to a patient in need of extracellular vesicles isolated from the plasma of oncological patients and loaded with a diagnostic and/or therapeutic drug.

### DETAILED DESCRIPTION OF THE INVENTION

According to a first object, the present invention describes extracellular vesicles comprising diagnostic and/or therapeutic drugs for the selective delivery to a tumor tissue.

For the purposes of this invention, the term “extracellular vesicles” (hereafter sometimes abbreviated as “EV”) is suggested to comprise all types of membrane vesicles released into the extracellular space, regardless of their differences in biogenesis and composition.

Therefore, exosomes, microvesicles, and apoptotic bodies are included within this definition.

In particular, exosomes are small vesicles (30-150 nm) involved in intercellular communication, microvesicles are vesicles of 100-1000 nm, while apoptotic bodies originate from apoptotic cells and their size is between 1000-5000 nm.

For the purposes of the present invention, a diagnostic drug is a drug chosen from the group which comprises:
fluorescent markers, e.g. selected from the group which comprises: DiD (1,1′-dioctadecyl-3,3,3′,3′-tetramethylindodicarbocyanine, 4-chlorobenzene sulfonate salt), ICG (indocyanine green, cardiogreen);
contrast media, e.g. selected from the group comprising compounds comprising gadolinium, such as gadoteric acid, gadodiamide, gadodenic acid, gadobutrol, gadofosveset, gadopentetic acid, gadoteridol and gadoxetic acid;
conjugated protein ligands, e.g. selected from the group comprising: ocreotide.

For the purposes of the present invention, a therapeutic drug is a tumor therapy drug and is preferably selected from the group which comprises:
chemotherapy drugs, e.g. selected from the group comprising: paclitaxel, gemcitabine, cisplatin, carboplatin, vinorelbine, pemetrexed;
monoclonal antibodies, e.g. selected from the group comprising: bevacizumab, cetuximab, nivolumab, pembrolizumab;
nucleic acids, e.g. selected from the group comprising: antisense oligonucleotides and aptamers;
oncolytic adenoviruses, e.g. selected from the group comprising Ad5D24.

According to a preferred aspect of the present invention, the described extracellular vesicles are isolated from blood plasma (hereinafter referred to as “plasma” for the sake of brevity).

According to a particularly preferred aspect of the present invention, the plasma is represented by the plasma of an oncological patient, i.e. a patient with an oncological pathology.

According to an aspect, the vesicles are isolated from the patient's own plasma (autologous use) to whom the vesicles are administered for diagnosis and/or cancer therapy, as reported below; alternatively, it is a different patient (heterologous use) from the one from whose plasma the vesicles are isolated.

For the purposes of the present patent application, the form of cancer from which the patient from whose plasma the extracellular vesicles are isolated is the same as the form of cancer from which the vesicles comprising the diagnostic or therapeutic drug are administered; alternatively, it is a different form of cancer.

In a preferred aspect of the present invention, the extracellular vesicles have a size between 50 and 300 nm.

According to another aspect of the invention, the vesicles have a zeta potential which is not modified by loading a diagnostic/therapeutic drug.

For the purposes of the present invention, the zeta potential is the net charge possessed by particles, i.e. the electrokinetic potential present in colloidal dispersions; in other words, the zeta potential is the potential difference between the dispersion medium and the stationary layer of fluid attached to the dispersed particle.

In a second and third object of the invention, a method for isolating and a process for purifying extracellular vesicles from the plasma of an oncological patient are described.

In particular, the method of the present invention comprises a step of preparing the plasma from an isolated sample of the patient's blood.

Such a step of preparing comprises, in particular, the steps of:

A1) treating an isolated blood sample of a patient with a suitable drug capable of inhibiting the coagulation cascade,

A2) subjecting the thus treated sample to a centrifugation step at low speed,

A3) separating the supernatant,

A4) subjecting the supernatant to a centrifugation step at high speed.

In particular, in step A1) the isolated blood is treated with ethylenediaminetetraacetic acid (EDTA) or alternatively with heparin or citrate.

For the purposes of the present invention, all subsequent steps must be carried out within a few hours of collection and preferably within 12 hours.

Step A2) of centrifugation is preferably carried out at the speed of 1600 RCF for a time of about 10 minutes at room temperature.

Step A4) of high-speed centrifugation is preferably carried out at a speed of about 3000 RCF for a time of about 10 minutes at room temperature.

Extracellular vesicles (EV) are thus isolated through the method as described above.

In a second and third object of the invention, a method for isolating and possibly also purifying extracellular vesicles from an isolated sample of plasma of an oncological patient are described.

In particular, the method for isolating comprises the steps of:

(B1) subjecting the isolated plasma sample to centrifugation,

B2) removing the supernatant.

More in detail, step B1) of centrifugation is preferably carried out at a speed of about 10,000 RCF for 120 minutes at a temperature of about 4° C.

Furthermore, step B1) is preferably carried out on an isolated plasma sample obtained as described above.

The solid deposit obtained from step B2) comprises extracellular vesicles, which can be resuspended in a suitable buffer solution.

If necessary, they can then be filtered.

In particular, said extracellular vesicles are resuspended in an appropriate buffer solution.

According to an aspect of the invention, if the extracellular vesicles are filtered, hydrophilic 0.1 μm mesh polytetrafluoroethylene (PTFE) filters are used to avoid contamination; e.g. phosphate buffer (PBS) containing bovine serum albumin (BSA), e.g. a concentration of approximately 0.5%, may be used.

For the purposes of the present invention, the suspension of extracellular vesicles in a buffer solution obtained as described above may be subjected to purification.

In a preferred aspect of the invention, said purification can be carried out by magnetic separation.

More in detail, if carried out, such purification comprises the steps of:

(C1) adding a solution containing magnetic beads bound to an appropriate antibody,

C2) incubating of the suspension of step C1) for an appropriate time,

C3) purifying in column by magnetic separation in a magnetic field,

C4) centrifuging the suspension of extracellular vesicles purified in step C3),

C5) removing the supernatant.

In step C1), a quantity of magnetic bead solution of about 20 μl may be added.

In particular, such magnetic beads are bound to an anti-human antibody CD81.

The incubation in step C2) is preferably carried out for 16 hours at 4° C.

More in detail, the column purification in step C3) comprises the steps of:

C3.a) loading the suspension onto a column for the magnetic separation in a magnetic field,

C3.b) washing with an appropriate solution to remove impurities. In a preferred aspect, the washing may be carried out with phosphate buffer solution followed by filtration according to the procedure in step B2,

C3.c) releasing extracellular vesicles by removing the magnetic field from the column and eluting with an appropriate solution. In a preferred aspect, the elution is carried out with a phosphate buffer solution, which is followed by appropriate filtration according to the procedure in step B2, and preferably at high pressure.

As regards to step C4), the centrifugation is preferably carried out at a speed of about 100,000 RCF for 120 minutes at a temperature of about 4° C.

After the removal of the supernatant, the vesicles are resuspended in an appropriate buffer solution, e.g. phosphate buffer (PBS), which is then filtered according to the procedure in step B2.

According to a particularly preferred aspect of the invention, the step of purification is not performed and, therefore, the process comprises only steps C4) and C5) above.

Therefore, according to such an aspect, the method proceeds with the steps of:

C1′) centrifuging the suspension of extracellular vesicles, and

C2′) removing the supernatant.

According to a fourth object of the invention, a method is described for loading drugs having diagnostic and/or therapeutic activity into extracellular vesicles isolated from the plasma of oncological patients.

In a preferred aspect, the loaded extracellular vesicles are obtained and possibly purified, according to the method of the present invention.

In particular, the method comprises the step D1) of incubating the extracellular vesicles for an appropriate time with a solution containing the diagnostic and/or therapeutic drug.

More specifically, the vesicles are incubated from a suspension comprising about 10⁸-10⁹ vesicles.

Preferably, such vesicles may be suspended in 1 ml of an appropriate buffer solution, e.g. phosphate buffer (PBS).

As described above, filtering, e.g. with hydrophilic, 0.1 μm mesh polytetrafluoroethylene (PTFE) filters, may follow.

The drug is incubated from a solution at an appropriate concentration, as a function of needs, e.g. such as the amount of drug to be delivered and as a function of the drug itself.

For the purposes of the present invention, the incubation is performed for a time of about 1 to 24 hours, preferably about 1 to 12 hours, as a function of needs, such as the amount of drug to be delivered and the nature of the drug itself.

The incubation is preferably carried out at 4° C.

After step D1) of incubating, the suspension can be subjected to the steps of:

D2) centrifuging, and

D3) removing the supernatant.

The extracellular vesicles thus loaded can be resuspended in an appropriate buffer solution, e.g. phosphate buffer (PBS), which is then filtered according to the procedure of step B2.

In particular, the step D2) is performed at a speed of about 150,000 RCF for a time of about 180 minutes at room temperature.

For the purposes of the present invention, given parameters of the method of loading the diagnostic and/or therapeutic drug depend on given factors, such as, for example: the volume of buffer solution to prepare the suspension to be centrifuged, the amount of incubation drug, the incubation time, the amount of solution for resuspension of extracellular vesicles loaded with the drug.

The identification of the precise conditions is considered to be within the expertise of a person skilled in the field.

The quantity will depend on the needs and the drug loaded inside the vesicles.

According to the fifth object of the invention, the medical use of the extracellular vesicles isolated from the plasma of oncological patients for the diagnosis and/or treatment of tumors is described.

In particular, as described above, the vesicles are isolated from the patient's own plasma to whom the vesicles are administered for diagnosis and/or cancer therapy (autologous use); alternatively, they are isolated from a patient's plasma to be subsequently used for medical use in a different patient (heterologous use).

For the purposes of the present patent application, the form of cancer from which the patient from whose plasma the extracellular vesicles are isolated is the same as the form of cancer from which the loaded vesicles are administered; alternatively, it is a different form of cancer.

The amount of preparation of loaded extracellular vesicles to be administered to the patient can be determined by the person skilled in the art based on needs.

In particular, the extracellular vesicles prepared according to the description above are administered intravenously.

In accordance with a further object describes a method for the diagnosis and/or therapy of tumors comprising the administration to a patient in need extracellular vesicles isolated from the blood plasma of oncological patients and loaded with a diagnostic and/or therapeutic drug as described above.

The type of drug and its quantity to be administered may be defined by the person skilled in the field as required.

In particular, the extracellular vesicles prepared according to the description above are administered intravenously.

The invention will be described further below by means of non-limiting examples.

### Example 1

### Preparation of Purified Extracellular Vesicles

A. Separation of Plasma from the Blood Sample
The blood (10-20 mL) is taken using vacuum tubes containing ethylenediaminetetraacetic acid (EDTA). The following steps must be carried out within a few hours of collection (maximum 12 hours).
The test tube (p1) is centrifuged at a speed of 1600 RCF for 10 minutes at room temperature (rt).
The supernatant is removed and transferred into a new test tube (p2) of adequate volume.
The test tube (p2) is centrifuged at a speed of 3000 RCF for 10 minutes at rt.
The supernatant is the plasma sample which will be used for the next steps.

### B. Isolation of Extracellular Vesicles

The plasma is transferred into test tubes for ultracentrifugation (U1).
The test tubes (U1) are centrifuged at a speed of 100000 RCF for 120 minutes at 4° C.
The supernatant is aspirated and eliminated.
The residual solid deposit in the test tubes (U1), containing the vesicles, is resuspended in 1 mL of phosphate buffer (PBS) containing 0.5% bovine serum albumin (BSA), suitably filtered with 0.1 μm filters.

### C. Purification of Extracellular Vesicles

20 μL of solution containing magnetic beads bound to an anti-human antibody CD81 is added to the suspension containing the vesicles.
The suspension of EV is incubated with the antibody for 16 hours at 4° C.
After incubation, the EV suspension is eluted through a column for magnetic field separation.
The column is washed 3 times with PBS, suitably filtered as described above.
The EVs inside the column are released by moving the column away from the magnetic field and washing it with 5 mL of PBS, suitably filtered as described above, at high pressure.
The suspension is transferred into a test tube for ultracentrifugation (U2).
The test tubes (U2) are centrifuged at a speed of 100000 RCF for 120 minutes at 4° C.
The supernatant is aspirated and eliminated.
The residual solid deposit in the test tubes (U2), containing the vesicles, is resuspended in 200 mL of phosphate buffer (PBS), suitably filtered as described above.

### Example 2

### Loading with Oncological Therapeutic Drug—Paclitaxel

A preparation of extracellular vesicles prepared and purified according to Example 1 is loaded with the oncological therapeutic drug Paclitaxel.
An amount comprised between 10⁸ and 10⁹ EV is resuspended in 1 mL of PBS, appropriately filtered as described above.
The therapeutic drug Paclitaxel is diluted to a concentration of about 10 nmol.
The EVs are incubated with the therapeutic drug for 1 hour at rt.
The sample is transferred into test tubes for ultracentrifugation (U3).
The test tubes (U3) are centrifuged at a speed of 150000 RCF for 180 minutes at rt.
The supernatant is aspirated and eliminated.
The residual solid deposit in the test tubes (U3) is resuspended in 1 mL of phosphate buffer (PBS), suitably filtered as described above.

### Example 3

### Loading with Diagnostic Drug—Indocyanine Green

An amount comprised between 10⁸ and 10⁹ EV is resuspended in 1 mL of PBS, appropriately filtered as described above.
10 μg of the diagnostic drug are diluted to an appropriate concentration.
The EVs are incubated with the diagnostic drug for 12 hours at 4° C.
The test tubes (U3) are centrifuged at a speed of 150000 RCF for 180 minutes at rt.
The supernatant is aspirated and eliminated.
The residual solid deposit in the test tubes (U3) is resuspended in 200 mL of phosphate buffer (PBS), suitably filtered as described above, for injection of the diagnostic.

### Example 4

### Loading with Biological Therapeutic Drug—Oncolytic Virus

An amount comprised between 108 and 109 EV is resuspended in 1 mL of PBS, appropriately filtered with 0.1 μm.
A preparation comprising 10⁹ of oncolytic virus is diluted to a concentration suitable for loading in EV.
The EVs are incubated with the biological drug for 1 hour at rt.
The sample is transferred into test tubes for ultracentrifugation (U3).
The test tubes (U3) are centrifuged at a speed of 150000 RCF for 180 minutes at rt.
The supernatant is aspirated and eliminated.
The residual solid deposit in the test tubes (U3) is resuspended in 1 mL of phosphate buffer (PBS).

Filtering with 0.1 μm filters may not be carried out.

### Example 5

### Vesicle Characterization Method

10 μL of the cell suspension obtained from Example 1 are taken for subsequent characterization.

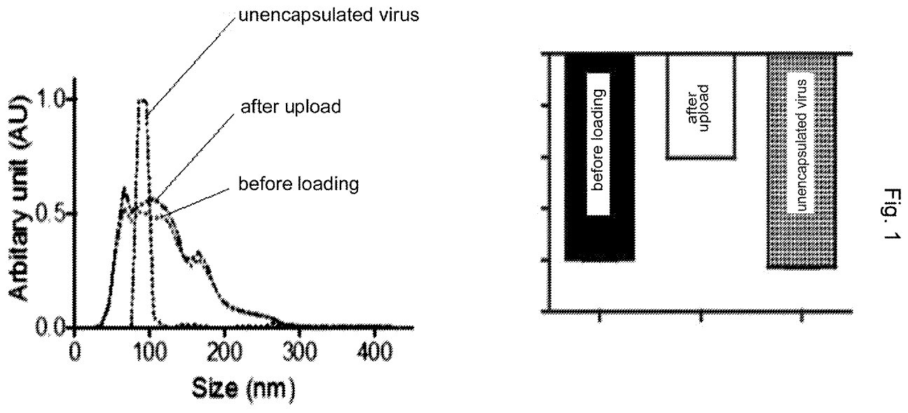

The number and size of the isolated EVs are determined using the Nanoparticle Tracking Analysis (NTA) (FIG. 1, left) and Electrophoretic Light Scattering (ELS, FIG. 1, right) following the indications of the instrument manufacturers (Miltenyi Biotec GmbH, Germany).

In particular, FIG. 1 shows the results of EV characterization obtained from plasma of oncological patients, before and after loading with therapeutic drugs.

The panel on the left shows the results of the dimensional analysis using the NTA technique: curves are related to vesicles before and after loading and the non-enveloped viruses. The results of the charge analysis using the ELS technique are shown in the panel on the right: left, the vesicles before loading, center, the vesicles after loading with the virus, right, the non-enveloped virus.

The vesicles to be used must have a size between 50 and 300 nm and a zeta potential which is not modified by the inclusion of therapeutic drugs (e.g. oncolytic virus in the figure).

### Example 6

### EV from Plasma from Oncological Patients as a Theragnostic Drug

A preparation of 1*10⁶ cells from a lung cancer cell line LL/2 (Lewis Lung carcinoma) was used for the test.

When the tumor has reached palpable size (diameter about 5 mm), basal autofluorescence emission was acquired to eliminate the “background noise”; the acquisition was obtained by gaseous anesthesia with diisoflurane and by measuring the fluorescence emission for 1 second of exposure through the IVIS Lumina II Quantitative Fluorescent and Bioluminescent Imaging device (PerkinElmer, Waltham, Mass., US).

The images are the basal fluorescence emitted by the animals (autofluorescence). The overlapping of reflected light and fluorescent images was done with Living Image Software 3.2 (PerkinElmer).

The EVs isolated from the plasma of oncological patients were loaded with an oncolytic virus as a therapeutic drug and with ICG as a diagnostic drug (EV1), as described in the procedure described above. 10⁸ EVs were injected intravenously and 24 hours after injection fluorescence emitted in vivo from rats (in vivo imaging) was acquired, as described above.

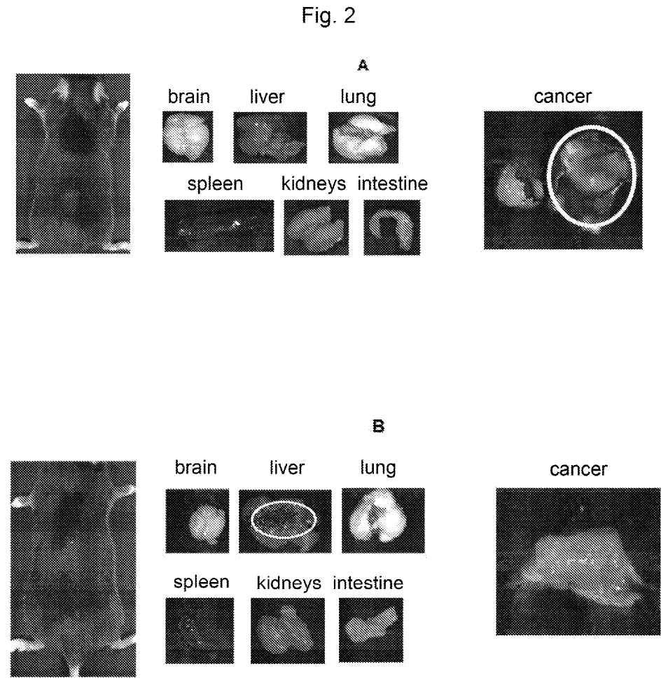

At the end of the acquisition, the rats were sacrificed by cervical dislocation, dissected, and the fluorescence emitted by the following organs was evaluated: brain, liver, spleen, kidneys, lungs, heart, intestine, adipose tissue, tumor tissue (e.g. see FIG. 2A) again using the IVIS Lumina II device. The in vivo result is shown in the upper panel of FIG. 2 and ex vivo result of the imaging fluorescence signal in the emission spectrum of Indocyanine green in the lower panel (ex.: 788 nm, em.: 813 nm). The animal underwent the fluorescence imaging procedure first in vivo and then ex vivo on the removed organs after sacrifice by cervical dislocation.

As shown in FIGS. 2A-B and 3A-B, the imaging experiments show a specific EV tropism from the plasma of oncological patients for tumor tissue. The injection of vesicles obtained from plasma of healthy individuals, and in particular not affected by oncological pathologies, loaded with an oncolytic virus, as a therapeutic drug, and with ICG, as a diagnostic drug (EV1), as described by the present invention and shown in the figures, did not show a specific accumulation in any of the examined organs, as determined using the described ex-vivo imaging procedure.

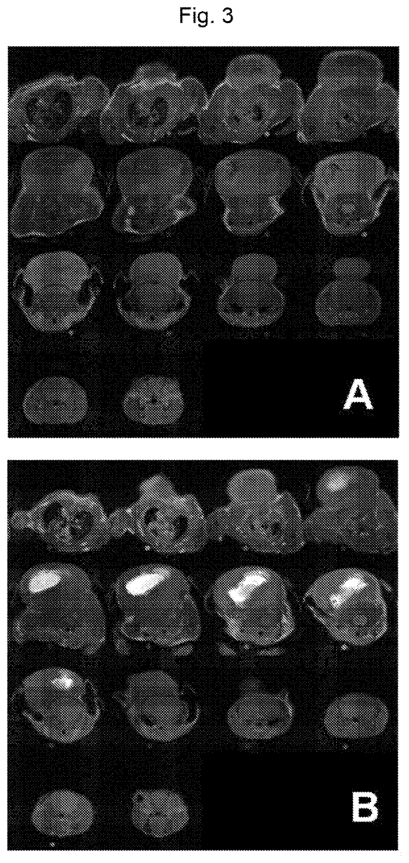

The IV injection in rats of 50 μL of an extracellular vesicle preparation obtained according to the present invention from an oncological patient and loaded with gadoteric acid showed a remarkable selectivity after 24 h after inoculation for LL2 tumor tissues (as shown in FIG. 3B) compared to the IV injection of 0.5 M of gadoteric acid 50 μL (FIG. 3A).

### Example 7

### Antibodies and Oligonucleotides

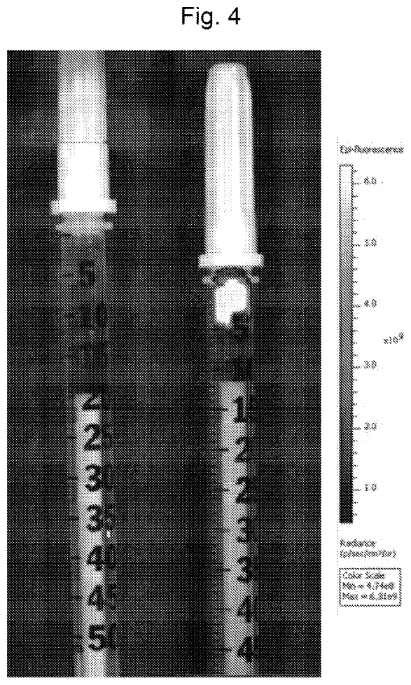

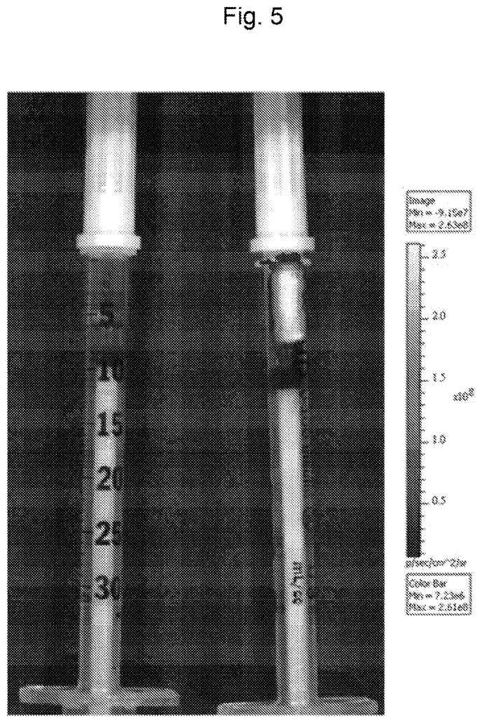

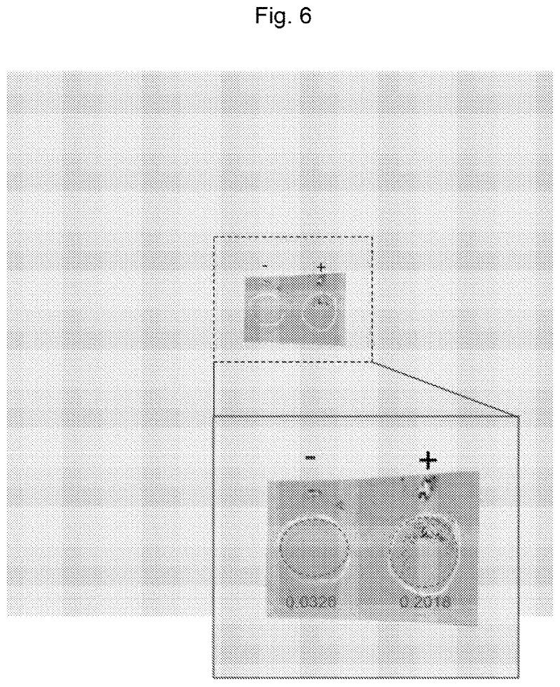

Assays for the incorporation of antibodies (FIGS. 4 and 5) and oligonucleotides (FIG. 6) were carried out.

In particular, for the assay in FIG. 4, fluorescence images were acquired with IVIS Lumina, applying ICG filters (ex. 710-760 nm; em. 810-875) on extracellular vesicles incubated for 10 minutes (left) or 12 hours (right) with ICG.

For the assay in FIG. 5, fluorescence images were acquired with IVIS Lumina, applying Cy5.5 filters (ex. 615-665 nm; em. 695-770) on extracellular vesicles incubated for 10 minutes (left) or 12 hours (right) with secondary anti-sheep antibody, conjugated with Alexafluor 647 fluorescent probe. FIG. 6 relates to the acquisition in chemiluminescence of dot blots related to extracellular vesicles incubated for 10 minutes (left, −) or 12 hours (right, +) with digoxygenin-conjugated oligonucleotides. The extracellular vesicles were treated with RIPA Lysis Buffer, spotted on PTFE membrane, and incubated with anti-DIG antibody, conjugated with HRP for 1 hour. After washing with TBS-T, the membrane was exposed to ECL and acquired with the Li-Cor Odyssey instrument. The magnification shows the densitometry values referring to the region of interest above (indicated with a dotted line).

### Example 8

### Use of EV from Oncological Patients to Mark the Tumor of Origin

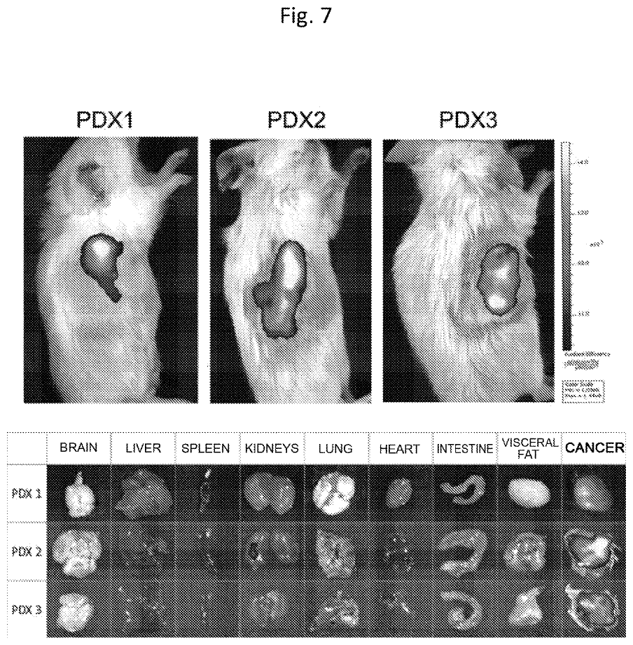

A xenograft derived from tumor tissue of the same patient from whose plasma the EVs were obtained was used for the test. Xenotransplantation is a study model where tissue or cells from a patient's tumor are implanted and allowed to proliferate in an immunodeficient rat, which is essential to prevent transplant rejection and promote its rooting. In detail, a volume of about 1 cm³ was taken from each selected nodule, cut into small fragments (about 3 mm³), then subcutaneously inoculated into the sides of SCID immunodeficient rats to an inoculated volume of about 100 mm³. When the tumor reached a size of 300-400 mm³ a basal autofluorescence emission acquisition was performed to eliminate the “background noise”; the acquisition was obtained through gaseous anesthesia with diisoflurane and by measuring the emission of fluorescence (ex:788 nm, em.: 813 nm) for 1 second of exposure through the IVIS Spectrum device (PerkinElmer, Waltham, Mass., US). The images obtained are the basal fluorescence emitted by the animals (autofluorescence) in the ICG fluorescence spectrum. The overlapping of reflected light and fluorescent images was done with Living Image Software 4.8 (PerkinElmer). The EVs isolated from the plasma of oncological patients were loaded as a therapeutic drug and with ICG as a diagnostic drug (EV1), as described in the procedure described above. 10⁸ EV1 were injected intravenously and 24 hours after injection fluorescence emitted in vivo from mice (in vivo imaging) was acquired, as described above. At the end of the acquisition, the rats were sacrificed by cervical dislocation, dissected, and the fluorescence emitted by the following organs was evaluated: brain, liver, spleen, kidneys, lungs, heart, intestine, adipose tissue, tumor tissue (e.g. see FIG. 7) again using the IVIS Spectrum device. The in vivo result is shown in the upper panel of FIG. 7 and ex vivo result of the imaging fluorescence signal in the emission spectrum of Indocyanine green in the lower panel (ex.: 788 nm, em.: 813 nm). The animal underwent the fluorescence imaging procedure first in vivo and then ex vivo on the removed organs after sacrifice by cervical dislocation. As shown in FIG. 7, the imaging experiments show a specific EV tropism from plasma from oncological patients for the tumor tissue originating from the carcinoma removed from the respective donor and implanted in the immunodeficient rat.

### Example 9

### Use of Autologous EV in Large Mammals (Dogs) for Diagnostic Purposes

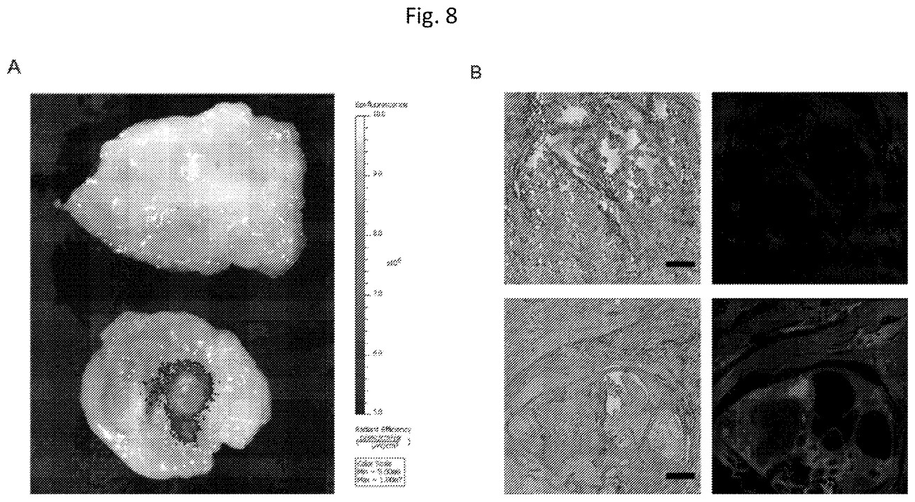

With the owners' informed consent, EVs were isolated from the blood of two dogs weighing 28 and 27 kg, admitted to a veterinary clinic for mastocytoma (i.e. superficial tumor originating from connective tissue mast cells) and mammary cancer, respectively. After isolation using the same procedure as described for EVs from human patient blood, the EVs were loaded with ICG as the diagnostic drug (EV1) as described in the procedure described above. The EVis were then intravenously re-infused to the donor dog 24 hours prior to surgery. In detail, 2-4*10⁶ EV/Kg were suspended in 10 ml of saline solution and injected at a rate of 1 ml/min. Immediately after surgical removal, the tumors were subjected to an imaging procedure using IVIS Lumina II Quantitative Fluorescent and Bioluminescent Imaging (PerkinElmer, Waltham, Mass., US), then fixed and included in paraffin for confirmation by NIR fluorescence microscopy and histology. Ex vivo imaging with the IVIS instrument revealed the presence of a fluorescent signal in the NIR spectrum only in the tumor area (FIG. 8A). In addition, microscopic fluorescence examination using NIR filters to detect ICG fluorescence revealed that while the samples collected in non-tumor tissue—i.e. tissue free of cancer cells—did not show a specific detectable fluorescent emission, the tumor samples showed fluorescence in the ICG spectrum (FIG. 8B), thus demonstrating that EVs isolated from blood using the procedure described and loaded with ICG can specifically mark autologous neoplastic tissue also in large mammals with spontaneous tumors. The hematological, liver, and kidney functions of the two dogs were determined before and after surgery without showing significant changes.

From the foregoing description, the advantages offered by the formulations of the invention will be apparent to the person skilled in the field.

In particular, as far as diagnostic applications are concerned, these can be:
intraoperative, or
in imaging diagnostics.

In intraoperative application, vesicles prepared according to this invention have the advantage of allowing a primary tumor or metastases to be visualized, delineating the margins of the tumor compared to healthy tissue and allowing the surgeon to operate precisely.

In imaging applications, the considered techniques most appropriate by the person skilled in the field can be used during preliminary diagnosis or treatment with a pharmacological or surgical drug.

In diagnostic imaging applications, moreover, the extracellular vesicles of the present invention allow the transport of a large amount of diagnostic drug due to their large volume; advantageously, this may lead to an increase in the sensitivity of the diagnostic method.

More in general, by virtue of the extracellular vesicles proposed by the present invention, drugs, represented by small molecules or biological agents, can be selectively delivered to the tumor target, thereby increasing the effectiveness of a therapeutic protocol and reducing its side effects.

By virtue of their size, the vesicles are very well suited to deliver large-sized drugs, large quantities of small-sized drugs, such as fluorescent molecules, chemical elements, such as gadolinium, radioactive molecules, such as 18FDG, or they can accommodate large-size molecules, such as antibodies, oligonucleotides or whole viruses.

In addition, it is worth noting that the extracellular vesicle acts as a shell protecting the drug from the action of metabolism and/or degradation performed by the human body, as well as protecting non-target tissues from the action of the drug itself; such an aspect is particularly important for biological drugs.

The process described for the preparation of the vesicles of the present invention loaded with drugs having therapeutic and/or diagnostic action comprises simple and fast steps and is, on the whole, a method which can be applied and integrated within the procedures of even the smallest hospital centers.

Furthermore, the use of autologous extracellular vesicles provides ample assurance of the absence of any rejection caused by incompatibility with host tissues.

What is claimed is:

1. Extracellular vesicles isolated from blood plasma of an oncological patient, said extracellular vesicles comprising a drug having diagnostic or therapeutic activity.

2. The extracellular vesicles isolated from blood plasma of an oncological patient according to claim 1, wherein said drug is a drug for tumor diagnosis or therapy.

3. The extracellular vesicles isolated from blood plasma of an oncological patient according to claim 1, wherein said drug is a drug for tumor diagnosis selected from the group consisting of:
fluorescent markers selected from the group consisting of: DiD (1,1′-dioctadecyl-3,3,3′,3′-tetramethylindodicarbocyanine, 4-chlorobenzene sulfonate salt), and ICG (indocyanine green, cardiogreen);
contrast media selected from the group consisting of gadolinium-comprising compounds; and
conjugated protein ligands selected from the group consisting of octreotide.

4. The extracellular vesicles isolated from blood plasma of an oncological patient according to claim 1, wherein said drug is a drug for tumor therapy selected from the group consisting of:
chemotherapy drugs selected from the group consisting of: paclitaxel, gemcitabine, cisplatin, carboplatin, vinorelbine, and pemetrexed;
monoclonal antibodies selected from the group consisting of: bevacizumab, cetuximab, nivolumab, and pembrolizumab;
nucleic acids selected from the group consisting of: antisense oligonucleotides and aptamers; and
oncolytic adenoviruses selected from the group consisting of Ad5D24.

5. The extracellular vesicles isolated from blood plasma of an oncological patient according to claim 1, wherein the diagnostic or therapeutic activity of said drug is directed towards the oncological patient from whom said extracellular vesicles are isolated or a patient different from the oncological patient from whom said extracellular vesicles are isolated.

6. The extracellular vesicles isolated from blood plasma of an oncological patient according to claim 1, wherein said extracellular vesicles have a size between about 50 and 300 nm.

7. The extracellular vesicles isolated from blood plasma of an oncological patient according to claim 1, wherein said extracellular vesicles comprise exosomes, microvesicles and apoptotic bodies.

8. A method for isolating extracellular vesicles from an isolated sample of blood plasma, said method comprising the steps of:
B1) subjecting said isolated sample of blood plasma to a centrifugation step at a speed of about 10,000 RCF for 120 minutes at a temperature of about 4° C., and
B2) removing the supernatant.

9. The method according to claim 8, further comprising a step of resuspending said extracellular vesicles in a suitable buffer solution and subjecting it to filtration.

10. The method according to claim 8, further comprising a step of purifying said extracellular vesicles.

11. A method for preparing an isolated sample of blood plasma from an oncological patient, said method comprising the steps of:
A1) treating said isolated sample of blood plasma with a suitable agent capable of inhibiting the coagulation cascade,
A2) subjecting the treated isolated sample of blood plasma to a centrifugation step at low speed,
A3) separating the supernatant, and
A4) subjecting the supernatant to a centrifugation step at high speed.

12. The method according to claim 11, wherein step A2) is carried out at a speed of 1,600 RCF for a time of about 10 minutes at room temperature.

13. The method according to claim 11, wherein step A4) is carried out at a speed of about 3,000 RCF for a time of about 10 minutes at room temperature.

14. A method for preparing extracellular vesicles isolated from blood plasma of an oncological patient comprising a drug having diagnostic or therapeutic activity, said method comprising incubating a suspension of extracellular vesicles obtained according to the method of claim 8 for a suitable period of time with a solution containing said drug having diagnostic or therapeutic activity.

15. The method according to claim 14, wherein a suspension comprising about 10⁸-10⁹ extracellular vesicles is incubated.

16. A diagnostic or therapeutic method comprising administering to a subject in need thereof, the extracellular vesicles isolated from blood plasma of an oncological patient according to claim 1.
